(19) United States
(12) Patent Application Publication
(10) Pub. No.: US 2022/0090000 A1
(43) Pub. Date: Mar. 24, 2022

(54) APPARATUS AND PROCESSES FOR ISOLATING BIOLOGICAL MATERIAL

(71) Applicant: UNIVERSITY OF WYOMING, Laramie, WY (US)

(72) Inventors: John OAKEY, Laramie, WY (US); John KISIDAY, Fort Collins, CO (US); Benjamin NOREN, Laramie, WY (US)

(21) Appl. No.: 17/484,381

(22) Filed: Sep. 24, 2021

Related U.S. Application Data
(60) Provisional application No. 63/082,979, filed on Sep. 24, 2020.

(51) Int. Cl. C12M 1/00; C12M 1/32; C12M 3/06; B01L 3/00

(52) U.S. Cl. CPC C12M 47/04; C12M 23/12; C12M 23/16; B01L 3/502707; B01L 2300/0829; B01L 2200/0647; B01L 2200/12; B01L 2300/123; B01L 3/502761

(57) ABSTRACT

Embodiments of the present disclosure generally relate to apparatus for isolating biological material, processes for fabricating such apparatus, and processes for using such apparatus. In an embodiment, an apparatus for isolating a biological material is provided. The apparatus includes a fluidic channel disposed over a portion of a substrate. The apparatus further includes a hydrogel structure disposed in the fluidic channel, the hydrogel structure comprising a plurality of wells, wherein each well of the plurality of wells has a diameter from about 1 μm to about 500 μm, the hydrogel structure comprising, in polymerized form, one or more photoreactive monomers.

### CROSS-REFERENCE TO RELATED APPLICATIONS

This application claims priority to U.S. Provisional Application No. 63/082,979, filed Sep. 24, 2020, which is incorporated herein by reference in its entirety.

### GOVERNMENT RIGHTS

This invention was made with government support under the Faculty Early Career Development Program (BBBE 1254608) awarded by the National Science Foundation and the Wyoming IDeA Networks of Biomedical Research Excellence program (P20GM103432) awarded by the National Institutes of Health. The government has certain rights in the invention.

### BACKGROUND

### Field

Embodiments of the present disclosure generally relate to apparatus for isolating biological material, processes for fabricating such apparatus, and processes for using such apparatus.

### Description of the Related Art

Interactions between cells and extracellular matrix (ECM) are responsible for directing vital cellular processes including migration, differentiation, and cell fate. Cell-ECM interactions are implicated in a number of pathologies and are a significant consideration in engineering and regenerating functional tissues. The difficulty in isolating and observing cell-ECM interactions from cell-cell interactions and other biological variables has made it challenging to probe cell-ECM phenomena.

Conventional methods for isolating and observing cells or other biological material rely heavily on the fabrication of microwell arrays and/or seeding cells or other biological material in individual microwells. Such methods are slow and tedious, and exhibit low throughput. Further, conventional methods for isolating and observing cells or other biological materials do not maintain cell and/or biological material viability for long periods of time to sufficiently probe cell-ECM interactions. Methods for fabricating the microwell arrays are also inefficient and costly as the microwell arrays are formed using stamp-molding of, e.g., agarose or gelatin. Further, the individual microwells of the microwell arrays typically have diameters (≥150 μm) that are too large to retain or trap single (or a very small number of) cells or other biological materials.

There is a need in the art for improved apparatus and processes for isolating biological materials that overcome these and other deficiencies.

### SUMMARY

Embodiments of the present disclosure generally relate to apparatus for isolating biological material, processes for fabricating such apparatus, and processes for using such apparatus.

In an embodiment, an apparatus for isolating a biological material is provided. The apparatus includes a fluidic channel disposed over a portion of a substrate. The apparatus further includes a hydrogel structure disposed in the fluidic channel, the hydrogel structure comprising a plurality of wells, wherein each well of the plurality of wells has a diameter from about 1 μm to about 500 μm, the hydrogel structure comprising, in polymerized form, one or more photoreactive monomers.

In another embodiment, a process for forming an apparatus for isolating a biological material is provided. The process includes introducing a reaction mixture to a first microfluidic channel, the reaction mixture comprising one or more photoreactive monomers and a photoinitiator. The process further includes polymerizing the reaction mixture using lithography, under polymerization conditions, to form a patterned hydrogel structure comprising a plurality of wells, the plurality of wells for isolating a cell, tissue, or other biological material.

In another embodiment, a process for isolating a biological material is provided. The process includes introducing a sample comprising a biological material to a hydrogel structure, wherein the hydrogel structure comprises, in polymerized form, one or more photoreactive monomers; and has a plurality of wells, one or more wells of the plurality of wells having a diameter from about 1 μm to about 500 μm, the one or more wells of the plurality of wells is configured to retain the biological material. The process further includes introducing a media to the hydrogel structure to remove a portion of the sample from the hydrogel structure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a schematic of an example apparatus for isolating a biological material according to at least one embodiment of the present disclosure.

FIG. 2A is a flowchart showing selected operations of an example process for forming a hydrogel structure having a plurality of features or microwells according to at least one embodiment of the present disclosure.

FIG. 2B is a flowchart showing selected operations of an example process for forming an apparatus for isolating a biological material according to at least one embodiment of the present disclosure.

FIG. 3 is a flowchart showing selected operations of an example process for isolating cells, tissues, or other biological materials according to at least one embodiment of the present disclosure.

FIG. 4A is an exemplary image of example polyethylene glycol norbornene-encapsulated mesenchymal stem cells (MSCs) according to at least one embodiment of the present disclosure.

FIG. 4B shows exemplary images of the isolation of MSCs by two example apparatus according to at least one embodiment of the present disclosure.

FIG. 4C is graph showing exemplary data of cell viability over one week for single unencapsulated MSCs in 40 μm microwells made with a 700 kilodalton (kDa) polyethylene glycol diacrylate (PEGDA), single unencapsulated MSCs in 40 μm microwells made with a 3400 kDa PEGDA, and single MSCs encapsulated in PEGNB, according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments of the present disclosure generally relate to apparatus for isolating biological material, processes for fabricating such apparatus, and processes for using such apparatus. The inventors have found new and improved apparatus and processes that enable control over various cellular microenvironment properties. Briefly, the apparatus includes an array of microwells in a fluidic channel that mimics in-vivo conditions. The apparatus includes, e.g., a plurality of features or microwells that are photolithographically-fabricated via digital light processing (DLP) from photoreactive monomers. The individual features or microwells are a cross-linked hydrogel mesh network that mimics the characteristics of the endogenous extracellular matrix (ECM).

The apparatus and processes described herein overcome deficiencies present in conventional apparatus. For example, the difficulty in isolating and observing cell-ECM interactions from cell-cell interactions and other biological variables using conventional apparatus has made it challenging to probe the cell-ECM phenomenon. In contrast, embodiments described herein enable isolation and observation of cell-ECM interactions. Here, the apparatus and processes described herein, can serve to isolate discrete, customizable quantities of biological material (e.g., cells, tissues, and/or other biological material). The isolation occurs in a rapid, massively parallelized, long-term manner within features or microwells of a hydrogel structure engineered to mimic endogenous tissue characteristics for decoupling cell and biological microenvironment interactions. The apparatus and processes for isolating biological material permit the biological material to maintain viability for long periods of time. As such, embodiments described herein enable a user to examine cell-cell interactions, cell-microenvironment interactions, cell-tissue interactions, tissue-tissue interactions, tissue microenvironment interactions, or combinations thereof.

The apparatus and processes additionally enable users to test and determine the preconditioned response of biological materials to added components such as added chemicals and/or biologics. Accordingly, the apparatus and processes described herein enable decoupling and control over cell/tissue microenvironment interactions and enable long-term, continuous data acquisition through imaging, physical, and chemical analyses not otherwise possible by conventional apparatus and methods.

While the present disclosure refers to “microwells,” it will be appreciated that the disclosure may be applied to wells having a smaller size (e.g., “nanowells”). The term “corral” and “microwell” may be used interchangeably.

Embodiments of the present disclosure generally relate to apparatus for e.g., isolating a biological material, observing short-term and/or long-term interactions of cell-cell and cell-microenvironment interactions, among other applications. In some embodiments, the apparatus can serve to rapidly isolate discrete, customizable quantities of cells, tissues, and/or other biological materials. The apparatus can recapitulate native biological structure and function such that the cells, tissues, and/or other biological materials are viable for long periods of time. Moreover, the apparatus can enable decoupling and controlling of cell/tissue microenvironment interactions for novel simulation of in-vitro conditions and allowing for long term, continuous data acquisition through imaging, physical, and chemical analyses not otherwise possible.

In some examples, the apparatus includes a plurality of features or microwells (e.g., about 100 or more, about 1000 or more, or about 10,000 or more features or microwells) that can be fabricated by photolithography. The features or microwells of the apparatus can function as “corrals” creating discrete environments in which to, e.g., isolate, observe, and direct cell/tissue behavior over long periods with precise control of environmental variables.

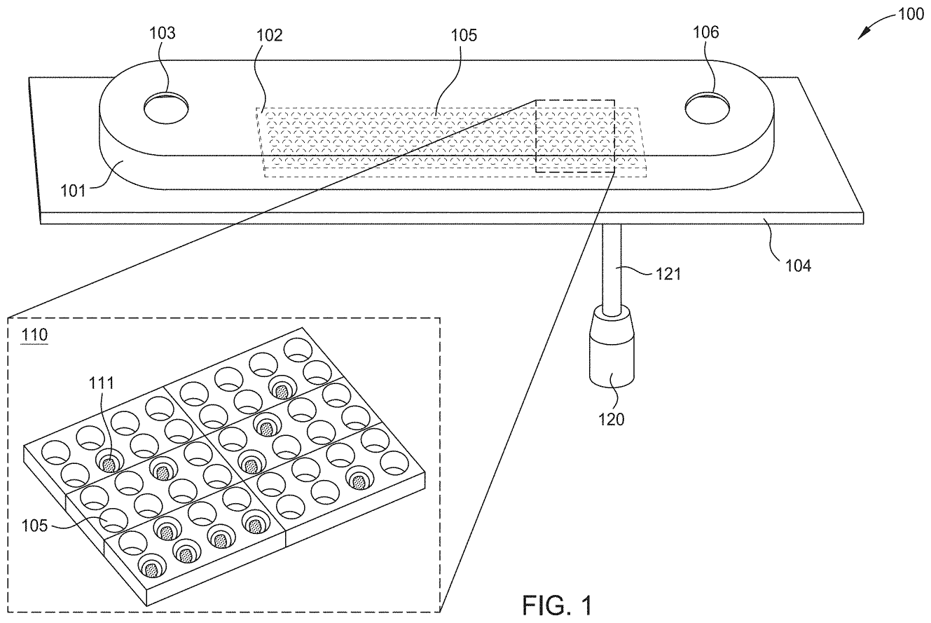

FIG. 1 is an example apparatus 100 according to at least one embodiment of the present disclosure. The apparatus is also referred to as a Biological Reconstruction of In-Vivo Conditions (BRIC) device. As stated above, the apparatus can be used for, e.g., isolating biological materials, and probing cell and matrix interactions as it mimics in-vivo conditions, among other uses. The biological materials that can be isolated by the apparatus 100 and subsequently probed include, but are not limited to, cells, tissues, other biological materials, or combinations thereof. In some embodiments, the apparatus 100 is a microfluidic device.

The apparatus 100 includes a fluidic channel 101. In at least one embodiment, the fluidic channel 101 has a diameter of micrometers (μm) to millimeters (mm), such as about 10 μm or more, such as from about 10 μm to about 2 mm, such as from about 50 μm to about 1 mm or from about 10 μm to about 1 mm. The apparatus 100 includes a port 103 (e.g., an accessible opening to the fluidic channel 101, such as a sample introduction port) where the sample containing the biological material, e.g., cells and/or tissues, is introduced. Typically, the sample that is introduced is in the form of a solution. In some embodiments, the size of the port 103 ranges from a few micrometers in diameter to a few millimeters in diameter depending on the application. The port 103 is coupled to the fluidic channel 101. An exit port 106 (e.g., an accessible exit from the fluidic channel 101) is coupled to the fluidic channel 101. The exit port 106 can allow for any remaining sample and/or solution to be removed from the fluidic channel 101. In some embodiments, the size of the exit port 106 ranges from a few micrometers in diameter to a few millimeters in diameter depending on the application. The port 103 and the exit port 106 can be made by utilizing a round punch tool to punch the port 103 and the exit port 106 through to the fluidic channel 101 so as to create an open pathway between the fluidic channel 101 and the exterior.

A hydrogel structure 102 is disposed within the fluidic channel 101. The hydrogel structure 102 includes a number features (e.g., microwells 105) that can serve to, e.g., isolate customizable quantities of biological material 111 (e.g., cells, tissues, or other biological materials). A single cell, a plurality of cells, a single tissue, a plurality of tissues, a single biological material, and/or a plurality of biological materials can be retained within, be held in, be trapped in, or otherwise be isolated within a single feature or a plurality of features, e.g., a single microwell 105 or a plurality of microwells 105. The features or microwells 105 can be patterned to desired shapes, sizes/dimensions, and/or morphologies in order to retain, hold, trap, or otherwise isolate a specific number of cells, tissues, and/or other biological materials. Further, the sizes or dimensions of the features or microwells 105 can be chosen to isolate cell(s), tissue(s), or other biological material(s) of a specific size, shape, chemical property, and/or physical property. By retaining, holding, trapping, or otherwise isolating cell(s), tissue(s), or other biological material(s), the features or microwells 105 of the apparatus 100 serve as corrals, creating discrete environments in which to observe and/or direct biological material behavior over long time periods with control over environmental variables.

The features or microwells 105 have a top surface that is open and a bottom surface that is closed or substantially closed. The bottom surface of the features or microwells 105 can be flat, substantially flat, angled, substantially angled, rounded, or substantially rounded.

The features or microwells 105 can have various shapes. Illustrative, but non-limiting, examples of shapes, include spherical, substantially spherical, rod-shaped, substantially rod-shaped, cylindrical, substantially cylindrical spiral, spiral, substantially spiral, comma-shaped, substantially comma-shaped, corkscrew-shaped, or substantially corkscrew-shaped. The shape of the features or microwells 105 can correspond to the shape of the cell(s), tissue(s), and/or other biological material(s) that is being isolated. For example, cocci have a spherical shape, bacilli have a rod shape, spirilla have a spiral shape, vibrios have a comma-shape, and spirochaetes have a corkscrew shape.

The hydrogel structure 102 can have a plurality of features or microwells 105 wherein a first portion of the plurality of features or microwells 105 have a first size/dimension, shape, and/or morphology that is different from a second portion of plurality of features or microwells 105 having a second size/dimension, shape, and/or morphology.

Inset 110 shows an enlarged view of the microwells 105 (or corrals) and biological material 111 within the features or microwells 105. The number, size/dimension, shape, and/or morphology of the features or microwells 105 can be tailored based on, e.g., the starting material(s) and initiators utilized to form the features or microwells 105, the reaction conditions of forming the features or microwells 105, the amount of time spent for the reaction, and/or the curing wavelength, among others. In some examples, the number of features or microwells 105 is about 100 or more, about 1000 or more, or about 10,000 or more, and/or about 50,000 or less, about 10,000 or less, about 1,000 or less, or about 500 or less. A higher or lower number of features or microwells 105 is contemplated.

In some embodiments, a diameter of an individual microwell is about 0.5 μm to about 1,000 μm, such as from about 50 μm to about 500 μm, such as from about 100 μm to about 450 μm, such as from about 150 μm to about 400 μm, such as from about 200 μm to about 350 μm, such as from about 250 μm to about 300 μm. In at least one embodiment, the diameter of the individual microwell ranges from diameter₁ to diameter₂ where each of diameter₁ to diameter₂ (in μm) is, independently, about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500, as long as diameter₁<diameter₂. Larger or smaller diameters are contemplated. The diameter of the individual microwell is measured at the top surface of the microwell using an Olympus IX-81 brightfield microscope with Metamorph® software.

In some embodiments, an opening of an individual feature is about 0.5 μm to about 1,000 μm, such as from about 50 μm to about 500 μm, such as from about 100 μm to about 450 μm, such as from about 150 μm to about 400 μm, such as from about 200 μm to about 350 μm, such as from about 250 μm to about 300 μm. In at least one embodiment, the opening of the individual feature ranges from diameter₁ to diameter₂ where each of opening₁ to opening₂ (in μm) is, independently, about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500, as long as opening₁<opening₂. Larger or smaller openings are contemplated. The opening of the individual feature is measured at the top surface of the feature using an Olympus IX-81 brightfield microscope with Metamorph® software.

Moreover, the features or microwells 105 can be fabricated in any combination of sizes. For example, a first portion of the microwells 105 of the hydrogel structure 102 can have a diameter that is different from a second portion of the microwells 105 of the hydrogel structure 102. As another example, a first portion of the features of the hydrogel structure 102 can have a size of opening that is different from a second portion of the features of the hydrogel structure 102. Example processes for forming the hydrogel structure 102 having features or microwells 105 are described below.

The fluidic channel 101 is affixed to a surface of a substrate 104 (e.g., a glass surface). It is contemplated that a material other than glass can be used as the substrate 104, such as plastics, elastomers, thermoplastics, polyethylene films, polyetheretherketone (PEEK) films, among others. In some embodiments, dimensions of the substrate are from about 1 mm×1 mm (about 1 mm²) to about 86 mm×54 mm (about 4,700 mm²), such as from about 10 mm² to about 4,500 mm², such as from about 50 mm² to about 4,000 mm².

FIG. 1 also shows a lithography device 120, such as a photolithography device for providing a source of light 121 to react components of the hydrogel forming solution in order to form the hydrogel structure 102. During use of the apparatus to isolate a biological material (as described below), the apparatus is free of the lithography device 120. Various lithography techniques are contemplated, such as lithography systems using masks or mask-less lithography systems (e.g., lithography based on DLP and digital mirror device (DMD) systems).

The lithography device 120 can be a digital light projector or a digital mirror device (such as a digital micromirror device), though other photolithography methods are contemplated. The digital light projector enables the projection of ultraviolet (and/or visible) light from a digital projector to flash an image of a designed pattern or a layer of a 3D model across the reaction mixture. The image projected corresponds to the desired hydrogel structure 102 having features or microwells 105. Upon projection of the ultraviolet (and/or visible) light components of the reaction mixture or hydrogel forming solution react to form, e.g., hydrogel structure 102 having features or microwells 105 in a desired pattern. This photolithography system is capable of automation to link an arbitrary number of individual features, pads of features (e.g., feature arrays), individual microwells and/or pads of microwells (e.g., microwell arrays) created in this manner.

The hydrogel structure 102 having a plurality of features or microwells 105 is formed from a reaction mixture that includes one or more photoreactive monomers, one or more linkers, one or more photoinitiators, other component(s), solvent(s), or combinations thereof. Other component(s) can include one or more cell adhesion peptides. This reaction mixture is interchangeably referred to as a hydrogel forming solution.

The one or more photoreactive monomers used to form the hydrogel contain photoreactive functional groups chemically attached to, e.g., polyethylene glycol (PEG). Illustrative, but non-limiting, examples of photoreactive functional groups include alkenes, thiols, acids, or combinations thereof. Upon irradiation, the photoreactive monomers (with or without co-reactants, such as linkers described below) can react to form a hydrogel.

Non-limiting examples of photoreactive monomers include, but are not limited to, polyethylene glycol norbornene (PEGNB), polyethylene glycol diacrylate (PEGDA), PEG methacrylate, polyethylene glycol di-photodegradable acrylate (PEGdiDPA), derivatives thereof, or combinations thereof. The photoreactive monomers can be branched (e.g., ˜20k 4-arm PEGNB and ˜40k 8-arm PEGNB) or unbranched. Other PEG-based derivatives having varied reactive functional groups are also contemplated. The molecular weight and shape (e.g., number of arms on PEGNB) of the one or more photoreactive monomers, among other characteristics, can be changed. Changing the molecular weight and shape of the photoreactive monomers (as well as the linker) can enable the tuning of various properties of the hydrogel structure 102, features and microwells 105, and can confer a range of traits to the system depending on the desired use and desired effect on cells, tissues, and/or other biological materials.

Photoreactive monomers can also include non-PEG-based monomers such as acrylates, acids (e.g., lactic acid, hyaluronic acid), gelatin, collagen, or combinations thereof. For example, polylactic acid (PLA), acrylated hyaluronic acid, gelatin methacrylate, derivatives thereof, and combinations thereof can be used. Block copolymers and triblock copolymers can also be used such as triblock PLA and PLA-PEG-PLA.

Molecular conformation of the photoreactive monomers can be varied to, e.g., impart desired material properties to the hydrogel microenvironment. For example, 1-arm molecular structures to 12-arm molecular structures can be used, such as 4-arm, 8-arm, or 12-arm molecular structures, such as 4-arm PEGNB, 8-arm PEGNB, 12-arm PEGNB, or combinations thereof.

Further, the chemical properties of the hydrogel microenvironment can be modified via click chemistry through addition of thiolated agents or similar acrylated agents such as thiolated or acrylated cell adhesion peptides like RGD (arginine-glycine-aspartate) or CRGDS (cystine-arginine-glycine-aspartate-serine). Mixtures of one or more photoreactive monomers, e.g., a mixture of PEGNB and PEGDA, can also be used, as well as mixtures that include non-PEG-based photolabile hydrogels such as gelatin methacrylate and/or photolabile hyaluronic acid.

A molecular weight of the one or more photoreactive monomers can be from about 100 Da to about 75,000 Da, such as from about 250 Da to about 50,000 Da, such as from about 5,000 Da to about 50,000 Da, such as from about 10,000 Da to about 45,000 Da, such as from about 15,000 Da to about 40,000 Da, such as from about 20,000 Da to about 35,000 Da, such as from about 25,000 Da to about 30,000 Da. Illustrative, but non-limiting, examples of the molecular weight of the photoreactive monomer are from about 250 Da to about 10,000 Da, such as from about 500 Da to about 9,000 Da, such as from about 1,000 Da to about 8,000 Da, such as from about 2,000 Da to about 7,000 Da, such as from about 3,000 Da to about 6,000 Da, such as from about 4,000 Da to about 5,000 Da. In some examples, the molecular weight of the one or more photoreactive monomers is 30,000 Da or less. In some embodiments, the molecular weight of the one or more photoreactive monomers ranges from MW₁ to MW₂ where each of MW₁ to MW₂ (in Da) is, independently, about 200, about 300, about 400, about 500, about 600, about 700, about 800, about 900, about 1,000, about 1,500, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, about 18,000, about 18,500, about 19,000, about 19,500, about 20,000, about 20,500, about 21,000, about 21,500, about 22,000, about 22,500, about 23,000, about 23,500, about 24,000, about 24,500, about 25,000, about 25,500, about 26,000, about 26,500, about 27,000, about 27,500, about 28,000, about 28,500, about 29,000, about 29,500, about 30,000, about 30,500, about 31,000, about 31,500, about 32,000, about 32,500, about 33,000, about 33,500, about 34,000, about 34,500, about 35,000, about 35,500, about 36,000, about 36,500, about 37,000, about 37,500, about 38,000, about 38,500, about 39,000, about 39,500, about 40,000, about 40,500, about 41,000, about 41,500, about 42,000, about 42,500, about 43,000, about 43,500, about 44,000, about 44,500, about 45,000, about 45,500, about 46,000, about 46,500, about 47,000, about 47,500, about 48,000, about 48,500, about 49,000, about 49,500, or about 50,000, as long as MW₁<MW₂. Higher or lower molecular weights of the one or more photoreactive monomers are contemplated. The molecular weight of the photoreactive monomer refers to the number average molecular weight (Ma). The Mₙ is the Mₙ provided by the manufacturer of the photoreactive monomer.

Suitable organic and/or aqueous solvents are utilized as a portion of the hydrogel forming solution. Such organic and/or aqueous solvents can include water, saline, phosphate buffered saline, appropriate biologically compatible liquid, or combinations thereof.

A concentration of the one or more photoreactive monomers in the hydrogel forming solution can be from about 5 wt % to about 75 wt %, such as from about 10 wt % to about 70 wt %, such as from about 15 wt % to about 65 wt %, such as from about 20 wt % to about 60 wt %, such as from about 25 wt % to about 55 wt %, such as from about 30 wt % to about 50 wt %, such as from about 35 wt % to about 45 wt %, based on a total weight percent of the components of the hydrogel forming solution (not to exceed 100 wt %). In at least one embodiment, the concentration of the one or more photoreactive monomers in the hydrogel forming solution is from about 5 wt % to about 35 wt %, such as from about 10 wt % to about 30 wt %, such as from about 15 wt % to about 25 wt %, based on the total weight percent of the components of the hydrogel forming solution (not to exceed 100 wt %). Higher or lower concentrations of the one or more photoreactive monomers can be used depending on application.

The components that are subjected to reaction can further include one or more linkers, such as a dithiol linker, such as a polyethylene glycol-dithiol (PEG-dithiol) linker, a derivative thereof, or combinations thereof. PEG-dithiol is a thiolated PEG having two thiol groups. The linker can be referred to as a thiol-containing monomer or dithiol linker unless the context indicates otherwise. When a dithiol linker is utilized, the photoreactive monomer(s) react with the thiol-containing monomer(s) via, e.g., a step-growth polymerization reaction occurring between the ene portion of the monomers and the thiol of the thiol-containing monomer.

A molecular weight of the one or more linkers (e.g., the PEG-dithiol linker) can be from about 500 Da to about 10,000 Da, such as from about 1,000 Da to about 9,500 Da, such as from about 1,500 Da to about 9,000 Da, such as from about 2,000 Da to about 8,500 Da, such as from about 2,500 Da to about 8,000 Da, such as from about 3,000 Da to about 7,500 Da, such as from about 3,500 Da to about 7,000 Da, such as from about 4,000 Da to about 6,500 Da, such as from about 4,500 Da to about 6,000 Da, such as from about 5,000 Da to about 5,500 Da. In some examples, the molecular weight of the linker is about 6,000 Da or less, such as from about 500 Da to about 6,000 Da, such as from about 1,000 Da to about 5,000 Da, such as from about 1,500 Da to about 4,500 Da, such as from about 2,000 Da to about 4,000 Da, such as from about 2,500 Da to about 3,500 Da. In some embodiments, the molecular weight of the one or more linkers ranges from MW₃ to MW₄ where each of MW₃ to MW₄ (in Da) is, independently, about 500, about 600, about 700, about 800, about 900, about 1,000, about 1,500, about 2,000, about 2,500, about 3,000, about 3,500, about 4,000, about 4,500, about 5,000, about 5,500, about 6,000, about 6,500, about 7,000, about 7,500, about 8,000, about 8,500, about 9,000, about 9,500, or about 10,000, as long as MW₃<MW₄. The molecular weight of the linker refers to the number average molecular weight (Mₙ). The Mₙ is the Mₙ provided by the manufacturer of the linker. Higher or lower molecular weights of the one or more linkers are contemplated. Illustrative, but non-limiting, examples of PEG-dithiol linkers include ˜1.5k PEG-dithiol, 3.5k PEG-dithiol, and ˜5k PEG-dithiol.

A concentration of the one or more linkers (e.g., PEG-dithiol) in the hydrogel forming solution can be from about 1 mM to about 50 mM, such as from about 5 mM to about 45 mM, such as from about 10 mM to about 40 mM, such as from about 15 mM to about 35 mM, such as from about 20 mM to about 30 mM, based on a total molar concentration of the components of the hydrogel forming solution. Higher or lower concentrations of the one or more linkers can be used depending on application.

The hydrogel forming solution can also include one or more photoinitiators. Illustrative, but non-limiting, examples of photoinitiators include lithium phenyl-2,4,6-trimethylbenzoylphosphinate (LAP) photoinitiator, 2-hydroxy-2-methyl propiophenone (e.g., Irgacure™ 1173, Darocur™ 1173), and combinations thereof. A concentration of the one or more photoinitiators in the hydrogel forming solution can be from about 0.0001 wt % to about 1 wt %, such as from about 0.001 wt % to about 0.9 wt %, such as from about 0.01 wt % to about 0.5 wt %, such as from about 0.05 wt % to about 0.1 wt %, based on the total wt % of the components of the hydrogel forming solution. Higher or lower concentrations of the one or more photoinitiators can be used depending on, e.g., the application or desired results.

The chemical properties of the hydrogel can be modified via click chemistry through addition of thiolated agents such as thiolated cell adhesion peptides like RGD or CRGDS. In some embodiments, the hydrogel forming solution can also include one or more cell adhesion peptides, such as thiolated cell adhesion peptides, such as RGD, CRGDS, or a combination thereof. A concentration of the one or more cell adhesion peptides in the hydrogel forming solution can be from about 0.5 mM to about 10 mM, such as from about 1 mM to about 8 mM, such as from about 2 mM to about 6 mM, such as from about 3 mM to about 4 mM based on the total molar concentration of the components of the hydrogel forming solution.

A non-limiting formulation useful for the hydrogel forming solution can include (a) from about 0.1 wt % to about 40 wt %, such as from about 1 wt % to about 40 wt %, such as from about 5 wt % to about 35 wt %, such as from about 10 wt % to about 20 wt % of one or more photoreactive monomers, such as a PEGNB, ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (b) from about 1 mM to about 100 mM, such as from about 5 mM to about 50 mM PEG dithiol ranging in molecular weight from about 100 Da to about 10,000 Da; and/or (c) from about 0.0001 wt % to about 1 wt %, such as from about 0.01 wt % to about 0.1 wt % of LAP photoinitiator. Additional components can be used as desired.

When PEGNB is utilized with a second photoreactive monomer such as PEGDA, PLA, PLA-PEG-PLA, etc., a non-limiting formulation can include the aforementioned formulation with about 0.1 wt % to about 40 wt %, such as from about 1 wt % to about 40 wt %, such as from about 5 wt % to about 35 wt %, such as from about 10 wt % to about 20 wt % of the second photoreactive monomer (e.g., PEGDA, PLA, PLA-PEG-PLA, etc.) having a molecular weight from about 1,000 Da to about 30,000 Da, such as from about 5,000 Da to about 20,000 Da, such as from about 10,000 Da to about 15,000 Da. Additional components can be used as desired.

An illustrative, but non-limiting, formulation useful to form a PEGPLA/NB composite hydrogels can include: (a) from about 0.1 wt % to about 40 wt %, such as from about 1 wt % to about 40 wt %, such as from about 5 wt % to about 35 wt % such as from about 10 wt % to about 20 wt % of a first photoreactive monomer (e.g., PLA-PEG-PLA, etc.) having a molecular weight from about 1,000 Da to about 30,000 Da, such as from about 5,000 Da to about 20,000 Da, such as from about 10,000 Da to about 15,000 Da; (b) from about 0.1 wt % to about 40 wt %, such as from about 1 wt % to about 40 wt %, such as from about 5 wt % to about 35 wt %, such as from about 10 wt % to about 20 wt % of a second photoreactive monomer (e.g., PEGNB, such as 4-arm PEGNB, 8-arm PEGNB, or a combination thereof) ranging in molecular weight from about 500 Da to about 50,000 Da, such as from about 3,000 Da to about 50,000 Da, such as from about 5,000 Da to about 20,000 Da, such as from about 10,000 Da to about 15,000 Da; (c) from about 1 mM to about 100 mM, such as from about 5 mM to about 50 mM PEG dithiol ranging in molecular weight from about 100 Da to about 10,000 Da; and/or (d) from about 0.0001 wt % to about 1 wt %, such as from about 0.01 wt % to about 0.1 wt % of the LAP photoinitiator.

Embodiments of the present disclosure also generally relate to processes for forming an apparatus, e.g., apparatus 100. Briefly, apparatus 100 can be made by temporarily bonding a first microfluidic channel to a substrate 104, forming the hydrogel structure 102 having features or microwells 105, replacing the first microfluidic channel with a second microfluidic channel that covers at least a portion (or all) of the features or microwells 105. The second microfluidic channel, e.g., fluidic channel 101, can be more strongly bonded to the substrate 104 than the first microfluidic channel. Embodiments of this process for forming the apparatus are further described below.

Embodiments of the present disclosure also generally relate to processes for forming a hydrogel structure, e.g., hydrogel structure 102 having features or microwells 105. Briefly, and in some embodiments, the process generally includes forming a reaction mixture that includes one or more photoreactive monomers, and then reacting the reaction mixture to form a hydrogel structure having a plurality of features or microwells, e.g., hydrogel structure 102 having a plurality of features or microwells 105. The hydrogel structure 102 can be a portion of an apparatus to isolate and probe cell(s) and/or other biological material(s) such as apparatus 100.

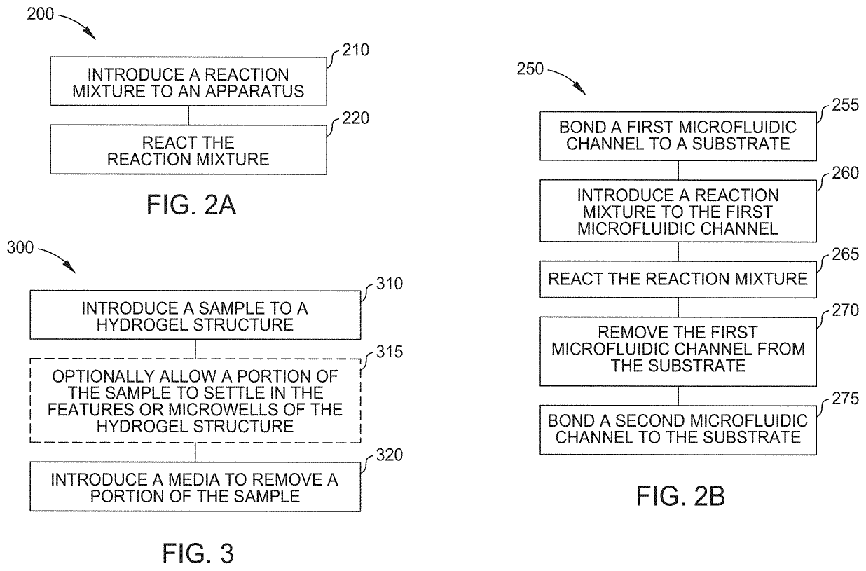

FIG. 2A is a flowchart showing selected operations of an example process 200 for forming a hydrogel structure having a plurality of features or microwells. The process 200 begins with introducing a reaction mixture (or hydrogel forming solution) to an apparatus at operation 210. In some examples, the apparatus is a microfluidic device having a channel in which the reaction mixture can flow. This channel can be a temporary channel, such as the first microfluidic channel described in relation to processes for forming the apparatus 100 (FIG. 2B). As described above, the reaction mixture (or hydrogel forming solution), can include one or more photoreactive monomers, one or more linkers, one or more photoinitiators, other components, and/or solvent(s).

Operation 210 can include flowing a hydrogel forming solution into a fluidic channel, which may be a temporary fluidic channel, at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. Higher or lower flow rates are contemplated for the hydrogel forming solution. Generally, any suitable flow rate to fill the fluidic channel can be utilized.

The process 200 further includes reacting the reaction mixture to form the hydrogel structure 102 having a plurality of microwells 105 at operation 220. The reaction can take the form of “click” chemistry, polymerization, click polymerization, and/or curing such that components of the reaction mixture react.

The reaction of operation 220 can be performed under reaction conditions, e.g., polymerization conditions. In some embodiments, the pH of the reaction mixture before, during, and/or after reaction can be from about 5 to about 9, such as from about 6 to about 8, such as from about 6.5 to about 7.5.

Reaction conditions of operation 220 can include exposing the reaction mixture to light (UV or visible) at a desired wavelength or wavelength range, such as a wavelength or wavelength range of about 290 nm to about 500 nm, such as from about 320 nm to about 460 nm, such as from about 340 nm to about 440 nm, such as from about 360 nm to about 420 nm, such as from about 380 nm to about 400 nm or from about 400 nm to about 420 nm, such as about 365 nm or about 405 nm, for varying timespans. In some embodiments, the wavelength or wavelength range of light is from about 290 nm to about 460 nm, such as from about 350 nm to about 450 nm, such as from about 375 nm to about 425 nm. The wavelength or wavelength range can be constant or varying during operation 220. It is contemplated that other wavelengths of light can be used with appropriate reacting photoinitiators.

The source of the light can be part of a lithography device 120 such as a digital light projector, though other photolithography methods are contemplated. The digital light projector enables the projection of an ultraviolet (and/or visible) light pattern from a digital projector to flash an image of a designed pattern or a layer of a 3D model across the reaction mixture. The image projected corresponds to the desired hydrogel structure having microwells. Upon projection of the ultraviolet (and/or visible) light pattern, the components of the reaction mixture or hydrogel forming solution react to form, e.g., hydrogel structure 102 having features or microwells 105 in a desired pattern. In some examples, the photolithography is performed utilizing a micro-digital light pattern projected through a microscope objective, enables control of both microwell size and shape.

The reaction conditions of operation 220 can further include a duration of exposure to the light. Such durations can be 1 millisecond (ms) or more and/or about 5 min. or less, such as from about 1 ms to about 60 seconds (s), such as from about 5 milliseconds to about 50 seconds, such as from about 50 milliseconds to about 45 seconds, such as from about 100 milliseconds to about 40 seconds, such as from about 0.5 seconds to about 30 seconds, such as from about 1 second to about 20 seconds. Shorter or longer durations of exposure to UV light are contemplated.

An energy density of the light for the reaction conditions of operation 220 can be from about 1 mW/cm² to about 10,000 mW/cm², such as from about 10 mW/cm² to about 1,000 mW/cm², such as from about 50 mW/cm² to about 500 mW/cm², such as from about 75 mW/cm² to about 150 mW/cm², such as from about 80 mW/cm² to about 120 mW/cm². Higher or lower energy densities are contemplated. The energy density can be constant or varying during operation 220.

The fabrication processes described can enable control of the physical, chemical, and mechanical characteristics of the apparatus's microenvironment by changing the size, shape, concentration, and number of reactive groups of the PEG-based and non-PEG-based monomer species. The processes additionally can enable control of these properties through data-informed adjustment of, e.g., the concentration of the components in the hydrogel forming solution and the UV light exposure duration and intensity. Adjusting these individual parameters, as well as others, can enable control of, e.g., the crosslinking density, pore size, and mechanical properties of the microwells.

As briefly described above, apparatus 100 can be made with the hydrogel structure 102 having features or microwells 105. FIG. 2B is a flowchart showing selected operations of an example process 250 for forming an apparatus, e.g., apparatus 100. The process 250 begins with bonding a first microfluidic channel to a substrate 104 at operation 255. The substrate can be made of transparent or a semi-transparent material, such as glass, silicon, plastics, elastomers, thermoplastics, polyethylene films, polyetheretherketone (PEEK) films, among others. Selection of the substrate 104 can be based on its transparency to UV and/or visible light. Here, and in some examples, operation 255 includes acrylating the substrate 104 and temporarily bonding the first microfluidic channel to the substrate 104 by known methods. The first microfluidic channel can be made of polydimethylsiloxane (PDMS). The PDMS self-adheres to form a water-tight, temporary bond. This temporary bond can be strengthened by, e.g., a short duration plasma treatment, and/or use of an aerosol adhesive.

When a plasma treatment is utilized to bond the first microfluidic channel to the substrate, plasma treatment can include placing the substrate 104 having the first microfluidic channel thereon in a plasma chamber. A pressure of oxygen in the plasma chamber can be set to about 100 mTorr to about 2 Torr, such as from about 200 mTorr to about 1 Torr, such as from about 300 mTorr to about 500 mTorr. The plasma can be struck at a suitable power level such as about 2 W to about 20 W, such as from about 5 W to about 15 W, such as about 8 W to about 12 W. The plasma treatment can be performed for a period of about 30 seconds to about 30 minutes, such as about 60 seconds to about 20 minutes, such as from about 90 seconds to about 15 minutes, such as from about 120 seconds to about 10 minutes, and at a temperature of about 15° C. to about 35° C., such as from about 20° C. to about 30° C. or from about 15° C. to about 25° C.

Process 250 further includes introducing a reaction mixture to the first microfluidic channel at operation 260 and reacting the reaction mixture at operation 265. The reaction of operation 265 results in a hydrogel structure 102 having features or microwells 105 of a desired design (e.g., size/dimension, shape, and/or morphology). Operations 260 and 265 can be the same as operations 210 and 220 of process 200. After forming the desired hydrogel structure 102, the first microfluidic channel can be removed at operation 270. The removal of the first microfluidic channel can be performed in any suitable manner, such as peeling off by hand or using a mechanical device such as tweezers or the like.

Process further includes bonding a second microfluidic channel to the substrate 104 at operation 275 by suitable methods. The second microfluidic channel can correspond to fluidic channel 101 of apparatus 100. The second microfluidic channel can be made of suitable materials such as PDMS, polyethylene terephthalate, glass, etc. The second microfluidic channel can cover at least a portion, or all, of the features or microwells 105. In some embodiments, the second microfluidic channel can have a larger diameter than the first microfluidic channel. The diameter of the second microfluidic channel can be chosen based on its desired use—e.g., the size of the cells, tissues, or biological materials that will pass through the apparatus 100 for, e.g., isolation.

Operation 275 can include the use of a plasma treatment to bond the second microfluidic channel to the substrate 104. The plasma treatment of operation 275 can be the same or similar to the plasma treatment described above for operation 255. The plasma treatment of operation 275 can be utilized to form a stronger bond between the substrate 104 and second microfluidic channel such that there is a more permanent attachment between the substrate 104 and the second microfluidic channel.

After operation 275, the apparatus 100 is generally formed. Holes for inlets and outlets (e.g., port 103 and exit port 106) can be formed in the second microfluidic channel, by suitable means, to enable introduction of a sample to the apparatus 100 and exit of a portion of the sample from the apparatus 100.

The apparatus and processes described herein enable the engineering of targeted endogenous tissue characteristics and isolation of single cells and providing superior control over the cell/tissue-microenvironment interface compared to conventional apparatus and methods. Furthermore, and relative to conventional methods, the processes described herein enable fabrication at significantly smaller resolutions at higher throughputs. In addition, the processes enable tuning of the material and chemical characteristics used to create the microwells such that the microwells possess targeted properties to probe the behavior of cells/tissues in a deliberate manner. Such tuning can be achieved by, e.g., click chemistry and lithography processing, to rapidly fabricate structures of varying sizes, shapes, and material properties, as described below. The apparatus formed by processes described herein can recapitulate biologically representative conditions in vitro due to, e.g., the properties of the hydrogel structure that isolates the biological material (e.g., cell(s), tissue(s) or other biological material).

Cells sense their microenvironment through specialized adhesion points, known as focal adhesions (FAs). FAs transmit biochemical data and enable cells to respond to their surroundings. Changing the aforementioned physical, chemical, and mechanical properties of the features or microwells of the hydrogel structure can enable a user to investigate and control cell adhesion, as well as mimic cell behavior in arbitrary tissues throughout the body by the aforementioned adjustment of polymer properties to match those of the target tissue. As described herein, the fabrication processes' control over, e.g., microwell shape, size, and/or chemical and physical properties, extends the ability to select a specific number of cells/tissues per microwell regardless of seeding density. Lithography such as photolithography using, e.g., DLP and/or a digital mirror device, enables control of both microwell size and shape. Using principles of, e.g., laminar fluid flow, the apparatus is able to select for a specific number of cells/tissues by the size, shape, and depth of the features or microwells.

Embodiments described herein also relate to uses of a hydrogel structure described herein for, e.g., isolating cells, tissues, and/or other biological materials. The number of cells that can be isolated using embodiments of the hydrogel structure 102 having features or microwells 105 can be any suitable number depending on the application. For example, the features or microwells 105 can be used to isolate a single cell or a plurality of cells (such as 2 or more, such as 3 or more). The features or microwells 105 can additionally, or alternatively, be used to isolate a single tissue, a plurality of tissues, a single biological material, and/or a plurality of biological materials. The number of cells, tissues, or other biological materials isolated by an individual feature or microwell 105 can be based on, e.g., the dimensions (such as diameter) of the features or microwells 105 and/or the dimensions/sizes of the cell, tissue, and/or other biological material that is to be isolated.

FIG. 3 is a flowchart showing selected operations of an example process 300 for isolating cell(s), tissue(s), and/or other biological material(s). The process 300 can utilize the hydrogel structure 102 having features or microwells 105 formed therein. In some embodiments, the process 300 utilizes apparatus 100.

The process 300 includes introducing a sample to the to the hydrogel structure 102 having microwells 105 at operation 310. The sample can include cells, tissues, and/or other biological materials in a suitable media such as water, saline, phosphate buffered saline, dulbecco's modified eagles media (DMEM), appropriate biologically compatible liquid (such as synovial fluid), other aqueous solution, or combinations thereof.

At operation 310, the sample can be introduced to the hydrogel structure 102 by using tubings coupled to the port 103 (e.g., introduction port) and the exit port 106 of apparatus 100. However, it is contemplated that introduction of the sample can be performed in other suitable ways, such as direct connecting Leuer lock type devices, snap-together microfluidic assemblies, and syringe-like devices, so as to introduce the sample without departing from the scope of the present disclosure. When using apparatus 100, and in some embodiments, the sample can be introduced to the hydrogel structure 102 via the port 103 which is coupled to the fluidic channel 101. Because the hydrogel structure 102 is disposed within the fluidic channel 101, the sample can travel through the port 103, the fluidic channel 101, the hydrogel structure 102, and exit the apparatus 100 via exit port 106. The sample containing the cells, tissues and/or other biological materials can be flowed into the fluidic channel 101 at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. In some embodiments, the flow rate for introducing the sample can be from about 1 μl/min to about 1000 μL/min, such as from about 50 μl/min to about 500 μl/min, such as from about 100 μl/min to about 400 μl/min, such as from about 200 μl/min about 300 μl/min. Higher or lower flow rates are contemplated.

In some examples, the sample includes cells in a suitable media such as water, saline, phosphate buffered saline, DMEM, appropriate biologically compatible liquid (such as synovial fluid), other aqueous solution, or combinations thereof. The cell(s) that can be retained within, held in, trapped in, or otherwise isolated in the features or microwells 105 include, but are not limited to, mesenchymal stem cells (MSCs), mesenchymal stromal cells, perinatal cells, fat derived stem cells, bone marrow aspirate concentrate, chondrocytes, regulatory T cells, and/or beta cells. It is contemplated that any other suitable cell can also be isolated. An amount of cells in the suitable media can be from about 1 cell/mL to about 1×10⁹ cells/mL, such as from about 1×10³ cells/mL to about 1×10⁸ cells/mL, such as from about 1×10⁵ cells/mL to about 1×10⁷ cells/mL. A higher or lower amount of cells in the suitable media can be utilized.

In some examples, the sample includes bacteria in a suitable media such as water, saline, phosphate buffered saline, DMEM, appropriate biologically compatible liquid (such as synovial fluid), other aqueous solution, or combinations thereof. The bacteria that can be retained within, held in, trapped in, or otherwise isolated in the features or microwells 105 include, but are not limited to, Listeria monocytogenes, Pseudomonas maltophilia, Thiobacillus novellus, Staphylococcus aureus, Streptococcus pyrogenes, Streptococcus pneumoniae, Escherichia coli, and Clostridium kluyveri. It is contemplated that any other suitable bacteria can also be isolated. An amount of bacteria in the suitable media can be from about 1 bacteria/mL to about 1×10⁹ bacteria/mL, such as from about 1×10³ bacteria/mL to about 1×10⁸ bacteria/mL, such as from about 1×10⁵ bacteria/mL to about 1×10⁷ bacteria/mL. A higher or lower amount of bacteria in the suitable media can be utilized.

In some examples, the sample includes tissue in suitable media such as water, saline, phosphate buffered saline, DMEM, appropriate biologically compatible liquid (such as synovial fluid), other aqueous solution, or combinations thereof. The tissue(s) that can be retained within, held in, trapped in, or otherwise isolated in the features or microwells 105 include, but are not limited to epithelial tissue, connective tissue, muscle tissue, and nervous tissue. An amount of tissue in the suitable media can be from about 1 unit of tissue/mL to about 1×10⁷ units of tissue/ml, such as from about 1×10² units of tissue/mL to about 1×10⁶ units of tissue/ml, such as from about 1×10³ units of tissue/mL to about 1×10⁵ units of tissue/ml. A higher or lower amount of tissue in the suitable media can be utilized.

The sample can include mixtures of cell types, mixtures of bacteria types, mixtures of tissue types, and/or mixtures of other biological materials.

During and/or after introduction of the sample to the hydrogel structure 102 (via the fluidic channel 101), cell(s), tissue(s), and/or other biological material(s) can be retained within, held in, trapped in, or otherwise isolated in the features or microwells 105 of the hydrogel structure 102. In some embodiments, the cell(s), tissue(s), and/or other biological material(s) can be allowed to settle into the microwells 105 at optional operation 315. For example, during and/or after introduction of the sample, the flow of the sample can be caused to stop for a suitable time period to allow the desired material(s) to settle into the microwells 105. Suitable time periods can be about 30 seconds or longer, such as about 5 minutes or less, such as about 1 hour or less. A longer or shorter time period is contemplated. Allowing the materials—cell(s), tissue(s), and/or other biological material(s)—can serve to aid the entry of such materials into the features or microwells 105, and can serve to aid retention, trapping, or isolation of such materials by the features or microwells 105.

In some embodiments, and during and/or after introduction of the sample to the hydrogel structure 102, the hydrogel structure and the sample can be heated at a temperature of about 20° C. to about 40° C., such as from about 25° C. to about 35° C.

In addition, a portion of the sample can also sit on or stick to the hydrogel structure 102, e.g., at a location peripheral to the features or microwells 105. This portion of the sample can be undesired materials that include cell(s), tissue(s), and/or other biological material(s) that are not desired to be isolated. This portion of cell(s), tissue(s), and/or other biological material(s) of the sample that are not retained within, not held in, not trapped in, or otherwise not isolated in the microwells 105 can be removed by introducing an appropriate media to flush the hydrogel structure 102 at operation 320.

The media utilized for flushing the hydrogel structure 102 at operation 320 can include, e.g., water, saline, phosphate buffered saline, DMEM, appropriate biologically compatible liquid (such as synovial fluid), other aqueous solution, or combinations thereof. At operation 320, the media utilized to remove a portion of the sample can be introduced to the hydrogel structure 102 by using tubings coupled to the port 103 (e.g., introduction port) and the exit port 106 of apparatus 100. However, it is contemplated that introduction of the media can be performed in other suitable ways, such as direct connecting Leuer lock type devices, snap-together microfluidic assemblies, and syringe-like devices, so as to introduce the sample without departing from the scope of the present disclosure. When using apparatus 100, and in some embodiments, the media can be introduced to the hydrogel structure 102 via the port 103 which is coupled to the fluidic channel 101. Because the hydrogel structure 102 is disposed within the fluidic channel 101, the media can travel through the port 103, the fluidic channel 101, the hydrogel structure, and exit the apparatus 100 via exit port 106. Flushing can be performed at a sufficient flow rate so as to remove excess cell(s), tissue(s), or other biological material(s) from the hydrogel structure 102. In some embodiments, the media utilized to remove the undesired material from the hydrogel structure 102 can be introduced to the fluidic channel 101 at a flow rate of about 0.1 μL/min to about 150 μL/min, such as from about 25 μL/min to about 125 μL/min, such as from about 50 μL/min to about 100 μL/min, such as from about 80 μL/min to about 100 μL/min. In at least one embodiment, the flow rate for flushing can be from about 1 μl/min to about 1000 μL/min, such as from about 50 μl/min to about 500 μl/min, such as from about 100 μl/min to about 400 μl/min, such as from about 200 μl/min about 300 μl/min. Higher or lower flow rates for the flushing media are contemplated.

The sample can be introduced one or more times to the hydrogel structure 102. Likewise, removal of the undesired portion of the sample (e.g., excess cell(s), tissue(s), or other biological material(s)) can be performed one or more times, such as before and/or after each sample introduction. The flow of the sample and/or the flow of the media used to remove the undesired portion of the sample can be caused to stop at any suitable time to, e.g., to allow portions of the sample to settle in the features or microwells 105 of the hydrogel structure 102 such that desired portions of the sample can be retained within, held in, trapped in, or otherwise isolated in the one or more individual features or microwells 105. For example, the flow of the sample can be caused to stop before and/or after one or more sample introductions, before and/or after the one or more flushings, or combinations thereof.

Movement of the sample and/or media from port 103 to the exit port 106 can be controlled by, e.g., laminar flow, capillary action, temperature, a pumping mechanism (e.g., a syringe pump, pressure pump, or piezoelectric pump), electrodes, and the like. Such elements controlling the movement can be placed at either opposing ends of the device, opposite ends, or along various regions along a length of the fluidic channel 101.

After the process 300 for isolating cells, tissues, or other biological materials is performed, an amount of cells, tissues, or other biological materials are retained within, held in, trapped in, or otherwise isolated in one or more individual features or microwells 105.

In some examples, the features or microwells 105 are designed to retain a targeted quantity of biological material (e.g., cells, tissues, and/or other biological materials) per feature or microwell 105. The amount of cells, tissues, or other biological materials retained within, held in, trapped in, or otherwise isolated in the one or more individual features or microwells 105 can be adjusted depending on, e.g., the size of the cell(s), tissue(s), and/or other biological material(s) of the sample, the physical/chemical properties of the cell(s), tissue(s), and/or other biological material(s) of the sample, the dimensions of the individual feature or microwell 105, and/or the chemical/physical properties of the hydrogel, among other variables.

For example, the features or microwells 105 can retain a desired number of biological material per microwell by adjusting the ratio of microwell diameter to retained biological material diameter. For example, a ratio of cell size to microwell diameter can be used to control the amount of cells to be retained within, held in, trapped in, or otherwise isolated in an individual microwell. As a non-limiting example, a microwell having a diameter of about 50 μm can retain, hold, trap, or otherwise isolate 1 cell or 2 cells having a size of 20 μm. As another non-limiting example, a microwell having a diameter of about 10 μm can retain, hold, or trap 1 biological material having a size of about 7 μm or about 3 biological materials having a size of about 3 μm. Such biological materials can be bacteria, which can range from a size of about 1-2 μm in diameter and about 5-10 μm in length. As another non-limiting example, a feature having an opening of about 50 μm can retain, hold, trap, or otherwise isolate 1 cell or 2 cells having a size of 20 μm. As another non-limiting example, a feature having a opening of about 10 μm can retain, hold, or trap 1 biological material having a size of about 7 μm or about 3 biological materials having a size of about 3 μm.

The apparatus with microwells or features containing the targeted quantity of biological material is capable of media replacement without dislodging the biological material and can be monitored and analyzed noninvasively via microscopy. The cell(s), tissue(s), and/or other biological material(s) retained within, held in, trapped in, or otherwise isolated in an individual feature or microwell 105 can be removed from the features or microwells 105 and collected, if desired, by, e.g., turning the apparatus 100 upside down and flushing with a media.

The following examples are put forth so as to provide those of ordinary skill in the art with a complete disclosure and description of how to make and use aspects of the present disclosure, and are not intended to limit the scope of aspects of the present disclosure. Efforts have been made to ensure accuracy with respect to numbers used (e.g. amounts, dimensions, etc.) but some experimental errors and deviations should be accounted for.

### EXAMPLES

### Example Process for Fabricating the Apparatus

Apparatus 100 is fabricated by the following example procedure. A glass coverslip was acrylated and a polydimethylsiloxane (PDMS) microfluidic channel was is temporarily bonded to the glass coverslip using appropriate established techniques. The glass coverslip and PDMS microfluidic channel are then cleaned with alcohol. When placed on the acrylated substrate, the PDMS microfluidic channel self-adheres to form a water-tight temporary bond.

The substrate bonded to the PDMS microfluidic channel was then flushed with aqueous silane-selective polylysine PEG to passivate the glass bottom and prevent biomaterial adhesion in cases where biomaterial adhesion is undesirable. The substrate bonded to the PDMS microfluidic channel was then then plasma treated by placing in a plasma chamber (e.g., Harrick Plasma Cleaner model PDC-001). The pressure of the chamber was placed at about 500 mTorr of pure oxygen and a plasma was struck using the medium power setting (about 10.2 W) for about 90 seconds at room temperature (about 15° C. to about 25° C.).

Two different hydrogel structures were formed based on the molecular weight of the photoreactive monomer PEGDA. The hydrogel forming solutions to fabricate the two hydrogel structures contained PEGDA (˜700 Da PEGDA or 3400 Da PEGDA), ˜0.3% lithium phenyl-2,4,6-trimethylbenzoylphosphinate (LAP) by mass, and an aqueous solution such as phosphate buffered saline. This solution is added to the microfluidic channel, and the microwells are fabricated via photolithography by micro-DLP (Mightex Polygon 400), or other equipment achieving analogous results. The UV light was set at about 405 nm and a power of about 200 mW/cm². The individual hydrogel forming solutions were exposed to the UV light for about 995 ms. The reaction afforded ˜40 μm diameter microwells for each hydrogel structure.

The microfluidic channel was removed by pealing it off of the glass by hand and replaced with a correspondingly deeper PDMS microfluidic channel covering the microwell-fabricated area to form the apparatus 100. This deeper channel was permanently attached via plasma treatment using the same or similar procedure to that described above.

PDMS forms a reversible, water-tight bond with glass when the two surfaces come in contact with each other. This reversible bond is strengthened by placing the glass-PDMS device in an oven set at about 100° C. for longer than about 3 hours. A permanent bond forms between glass and PDMS when plasma treated because the PDMS surface is molecularly altered to be similar to the glass surface. When the plasma treated PDMS comes into contact with glass, such as a glass microscope slide, it forms an irreversible glass-glass bond.

Typically, the deeper channel is a minimum of about 30 μm deeper than the shallow channel to allow cells to pass through. An introduction port (e.g., port 103) and an exit port (e.g., exit port 106) were formed using a round punch tool so as so as to create an open pathway between the fluidic channel 101 and the exterior.

### Example Process for Using the Apparatus

Unencapsulated equine marrow-derived mesenchymal stromal cells (MSCs) in a solution of 100% synovial fluid and incubated at about 37° C. were seeded in the two different apparatus—the apparatus having a hydrogel structure made from 700 Da PEGDA and the apparatus having a hydrogel structure made from 3400 Da PEGDA. The biological material was then allowed to settle for less than about 30 minutes. Phosphate buffered saline or synovial fluid was then flushed through the apparatus 100 via fluidic channel 101, and excess biological material is washed away, exiting through exit port 106. As a comparative example (C.Ex. 1), equine marrow-derived MSCs were encapsulated in 8.2% PEGNB hydrogel microspheres of ˜120 μm diameter using a microfluidic droplet generator. The PEGNB-encapsulated MSCs were seeded under the same conditions in a 10 μL straight microfluidic channel. Table 1 shows a description of the examples and the comparative example.

TABLE 1

| Sample | Description |
|---|---|
| Ex. 1 | Unencapsulated MSCs in ~40 μm diameter microwells |
| | made of 700 Da PEGDA |
| Ex. 2 | Unencapsulated MSCs in ~40 μm diameter microwells |
| | made of 3400 Da PEGDA |
| C. Ex. 1 | PEGNB-encapsulated MSCs |

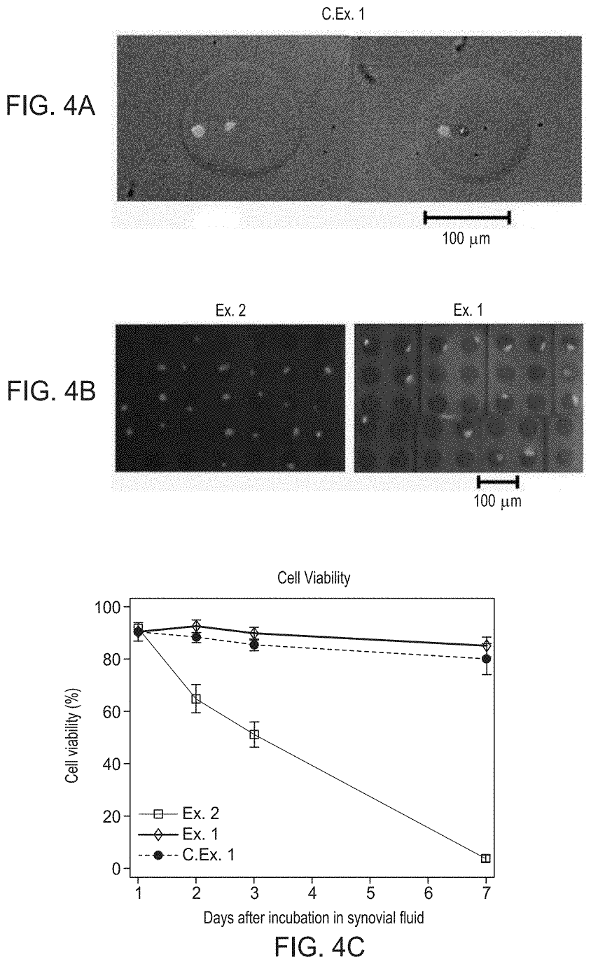

FIG. 4A shows fluorescent images of the comparative example PEGNB-encapsulated MSCs where the cells fluoresce green. The images indicate that the MSCs are encapsulated in the PEGNB hydrogels.

The MSCs of Ex. 1, Ex. 2, and C. Ex. 2. were observed for cell viability over one week. The microwells made from the 700 Da PEGDA (Ex. 1) provided a stiff attachment environment for the single unencapsulated MSCs. As shown in FIG. 4B (right panel, Ex. 1), the MSCs migrated to the microwells, attached to the microwells, and changed from round to flat, taking the form of a “fried egg” morphology within about 2 days. This morphology change is indicative of attached MSCs. Stem cells exhibiting this behavior in 700 Da PEGDA microwells also demonstrated excellent viability. As shown in FIG. 4B (left panel, Ex. 2), the ˜40 μm diameter microwells made from the 3400 Da PEGDA (Ex. 2) provided softer conditions for the MSCs such that the cells moved around the microwells extensively with very limited attachment.

FIG. 4C is a graph showing exemplary data of cell viability over one week for Ex. 1, Ex. 2, and C.Ex. 1. MSCs maintained greater than about 90% viability after 1 week in the hydrogel structure of Ex. 1. Similarly, the PEGNB-encapsulated MSCs (C.Ex. 1) showed attachment over 1 week. MSCs encapsulated in PEGNB typically do not change morphology due to the 3D space they inhabit. The MSCs for Ex. 2 showed good cell viability on day 1 and 2, but after one week, the MSCs maintained about 1% viability.

The examples shows that cells can maintain excellent viability over long periods of time, demonstrating favorable cellular microenvironment conditions. Overall, the results show that mechanical conditions can impact cell migration and cell viability, and that the microwell apparatus described herein maintains cell viability. In addition, the processes described herein enable the creation of features or microwells possessing tunable materials and chemical properties at single-cell resolution and high throughputs. The control and creation of such characteristics are not possible with conventional methods. Moreover, the processes and apparatus described herein provide an avenue to probe biological material, e.g., cell and/or tissue, behavior in a deliberate manner. It achieves this through a process employing hydrogel click chemistry and digital light projection photolithography to rapidly fabricate structures of arbitrary size, shape, and material properties in a “on chip” microfluidic system that together allows for imaging, physical, and chemical analysis over time.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, embodiments comprising “a microwell” include embodiments comprising one, two, or more microwells, unless specified to the contrary or the context clearly indicates only one microwell is included.

The term “coupled” is used herein to refer to elements that are either directly connected or connected through one or more intervening elements.

While the foregoing is directed to embodiments of the present disclosure, other and further embodiments of the disclosure may be devised without departing from the basic scope thereof, and the scope thereof is determined by the claims that follow.

What is claimed is:

1. An apparatus for isolating a biological material, comprising:
a fluidic channel disposed over a portion of a substrate; and
a hydrogel structure disposed in the fluidic channel, the hydrogel structure comprising a plurality of wells, wherein each well of the plurality of wells has a diameter from about 1 μm to about 500 μm, the hydrogel structure comprising, in polymerized form, one or more photoreactive monomers.

2. The apparatus of claim 1, wherein one or more wells of the plurality of wells has a diameter from about 30 μm to about 120 μm.

3. The apparatus of claim 1, wherein one or more wells of the plurality of wells has a diameter from about 5 μm to about 20 μm.

4. The apparatus of claim 1, wherein one or more wells of the plurality of wells are configured to retain a cell, tissue, other biological material, or combinations thereof, for about 24 hours or more.

5. The apparatus of claim 1, wherein the one or more photoreactive monomers comprise a methylene functional group, an acid functional group, or combinations thereof.

6. The apparatus of claim 5, wherein, when the one or more photoreactive monomers comprise the methylene functional group, the one or more photoreactive monomers comprise polyethylene glycol norbornene, polyethylene glycol diacrylate, derivatives thereof, or combinations thereof.

7. The apparatus of claim 5, wherein, when the one or more photoreactive monomers comprise the acid functional group, the one or more photoreactive monomers comprise polylactic acid, derivatives thereof, or combinations thereof.

8. The apparatus of claim 1, wherein the one or more photoreactive monomers comprises polyethylene glycol diacrylate, polyethylene glycol norbornene, polyethylene glycol methacrylate, polyethylene glycol di-photodegradable acrylate, acrylated hyaluronic acid, gelatin methacrylate, polylactic acid, or combinations thereof.

9. The apparatus of claim 1, wherein the hydrogel structure further comprises, in polymerized form, one or more thiol linkers.

10. The apparatus of claim 9, wherein the one or more thiol linkers is a polyethylene glycol dithiol linker.

11. The apparatus of claim 9, wherein:
the one or more thiol linkers has a molecular weight from about 500 Da to about 10,000 Da;
the one or more photoreactive monomers has a molecular weight from about 250 Da to about 50,000 Da; or
a combination thereof.

12. A process for forming an apparatus for isolating a biological material, comprising:
introducing a reaction mixture to a first microfluidic channel, the reaction mixture comprising one or more photoreactive monomers and a photoinitiator; and
polymerizing the reaction mixture using lithography, under polymerization conditions, to form a patterned hydrogel structure comprising a plurality of wells, the plurality of wells configured to isolate a cell, tissue, or other biological material.

13. The process of claim 12, further comprising:
bonding the first microfluidic channel to a substrate prior to introducing the reaction mixture to the first microfluidic channel;
removing the first microfluidic channel from the substrate after forming the patterned hydrogel structure; and
bonding a second microfluidic channel to the substrate such that the second microfluidic channel covers at least a portion of the patterned hydrogel structure.

14. The process of claim 12, wherein the one or more photoreactive monomers comprises polyethylene glycol diacrylate, polyethylene glycol norbornene, PEG methacrylate, polyethylene glycol di-photodegradable acrylate, acrylated hyaluronic acid, gelatin methacrylate, polylactic acid, or combinations thereof.

15. The process of claim 12, wherein:
a molecular weight of the one or more photoreactive monomers is from about 250 Da to about 50,000 Da;
the reaction mixture further comprises a thiol linker having a molecular weight of about 10,000 Da or less;
a diameter of the plurality of wells is from about 1 μm to about 500 μm; or
combinations thereof.

16. The process of claim 12, wherein the polymerization conditions comprise:
exposing the one or more photoreactive monomers to ultraviolet (UV) light;
a duration of exposure to the ultraviolet light that is from about 1 millisecond to about 60 seconds; and
an energy density of the ultraviolet light that is from about 1 mW/cm² to about 10,000 mW/cm².

17. The process of claim 12, wherein a diameter of the plurality of wells is from about 5 μm to about 100 μm.

18. A process for isolating a biological material, comprising:
introducing a sample comprising a biological material to a hydrogel structure, wherein the hydrogel structure:
comprises, in polymerized form, one or more photoreactive monomers; and
has a plurality of wells, one or more wells of the plurality of wells having a diameter from about 1 μm to about 500 μm, the one or more wells of the plurality of wells is configured to retain the biological material; and
introducing a media to the hydrogel structure to remove a portion of the sample from the hydrogel structure.

19. The process of claim 18, wherein the one or more wells of the plurality of wells is configured to retain a single cell.

20. The process of claim 18, wherein the one or more wells of the plurality of wells has a diameter of about 5 μm to about 200 μm.
